(19) United States
(12) Patent Application Publication
(10) Pub. No.: US 2013/0096401 A1
(43) Pub. Date: Apr. 18, 2013

(54) WIRELESS DISPOSABLE SHOCK TRAUMA MONITORING DEVICE

(71) Applicants: Robert Evan Lash, Redwood City, CA (US); Jian-Min Mao, Fremont, CA (US); Barbara Sue Faries, Fremont, CA (US)

(72) Inventors: Robert Evan Lash, Redwood City, CA (US); Jian-Min Mao, Fremont, CA (US); Barbara Sue Faries, Fremont, CA (US)

(21) Appl. No.: 13/651,036

(22) Filed: Oct. 12, 2012

Related U.S. Application Data
(60) Provisional application No. 61/546,664, filed on Oct. 13, 2011.

(51) Int. Cl. A61B 5/1455

(52) U.S. Cl. 600/323

(57) ABSTRACT

Apparatus for monitoring oxygen saturation levels in tissue for a miniature wireless disposable optical tissue oximeter to are disclosed. According to one aspect of the present invention, a sensor contains a first light source, a second light source, a photodetector, and a skin contact detector. Once skin contact is detected, the first light source emits light in the near infrared region, and the second light source emits light in the visible red region. The emitted light passes through a transparent layer of an adhesive fixation unit, and enters the underlying tissue, where a portion of the light is absorbed by tissue chromophores, including oxygenated hemoglobin and deoxygenated hemoglobin, and reflected back out of the tissue into the photodetector. The oxygen saturation of the tissue under the sensor is then calculated. The oxygen saturation measurements are wirelessly transmitted to a remote display device, such as a smartphone running a smartphone software application which receives the measurements and displays them in numeric, graphical, and audible form. In addition, the smartphone software application may relay the data to the Internet for remote viewing on a web site or remote transfer to a hospital patient data system.

### CROSS REFERENCE TO RELATED APPLICATIONS

This application claims the benefit under 35 U.S.C. §119(e) of U.S. Provisional Application No. 61/546,664, filed Oct. 13, 2011, the disclosure of which is hereby incorporated herein by reference in its entirety.

### BACKGROUND OF THE INVENTION

1. Field of Invention

The present invention relates generally to optical systems that monitor oxygen levels in tissue. More specifically, the present invention relates to a miniature wireless disposable optical tissue oximeter to monitor oxygen levels in tissue for use in shock trauma and exercise training applications.

2. Description of the Related Art

Near-infrared spectroscopy has been used for non-invasive measurement of various physiological properties in animal and human subjects. The basic principle underlying the near-infrared spectroscopy is that physiological tissues include various highly-scattering chromophores to the near-infrared waves with relatively low absorption. Many substances in a medium may interact or interfere with the near-infrared light waves propagating therethrough. Human tissues, for example, include numerous chromophores such as oxygenated hemoglobin, deoxygenated hemoglobin, water, lipid, and cytochrome, where the hemoglobins are the dominant chromophores in the spectrum range of approximately 700 nm to approximately 900 nm. Accordingly, the near-infrared spectroscope has been applied to measure oxygen levels in the physiological medium such as tissue hemoglobin oxygen saturation and total hemoglobin concentrations.

Various techniques have been developed for the near-infrared spectroscopy, e.g., time-resolved spectroscopy (TRS), phase modulation spectroscopy (PMS), and continuous wave spectroscopy (CWS). In a homogeneous and semi-infinite model, both TRS and PMS have been used to obtain spectra of an absorption coefficient and reduced scattering coefficient of the physiological medium by solving a photon diffusion equation, and to calculate concentrations of oxygenated and deoxygenated hemoglobins as well as tissue oxygen saturation. CWS has generally been designed to solve a modified Beer-Lambert equation and to measure changes in the concentrations of oxygenated and deoxygenated hemoglobins.

Despite their capability of providing the hemoglobin concentrations as well as the oxygen saturation, one major drawback of TRS and PMS is that the equipment is bulky and expensive. CWS may be manufactured at a lower cost but limited in its utility because it cannot compute the oxygen saturation from the changes in the concentrations of oxygenated and deoxygenated hemoglobins.

Optical Diffusion Imaging and Spectroscopy (ODIS) allows tissue to be characterized based on measurements of photon scattering and absorption. In tissue such as human tissue, near infrared light is highly scattered and minimally absorbed. Optical diffusion imaging is achieved by sending optical signals into tissue and measuring the corresponding diffuse reflectance or transmittance on the tissue surface.

Scattering is caused by the heterogeneous structure of a tissue and, therefore, is an indicator of the density of a cell and the nuclear size of the cell. Absorption is caused by interaction with chromophores. ODIS emits light into tissue through a sensor. The position of the light source which emits the light and a detector which detects the light allows a depth of measurement to be determined. A ratio of oxyhemoglobin and deoxyhemoglobin may be used to allow for substantially real-time measurement of oxygen, e.g., oxygen saturation levels.

The measurement of oxygen saturation levels in tissue has proven useful in a number of application areas, including the assessment of trauma patients who may experience a loss of circulatory volume due to internal or external bleeding. As blood loss progresses, the body initially compensates by shifting blood out of the limbs, by means of peripheral vasoconstriction, into the central circulation to preserve blood flow to the brain and to the internal organs. Peripheral vasoconstriction causes a drop in the measured peripheral tissue oxygen saturation. Early detection of this drop in tissue oxygen saturation allows for early intervention. As blood loss progresses further, the body additionally compensates by increasing the heart rate in an attempt to maintain normal blood pressure for perfusing the brain and internal organs. When blood loss becomes extreme, these compensatory mechanisms are no longer sufficient and the blood pressure falls, resulting in a shock state. Blood perfusion to organs is then impaired, and can result in stroke and permanent organ injury known as Multiple Organ Dysfunction Syndrome (MODS) leading to complications such as kidney failure, liver failure, and ischemic bowel infarction. The mortality rate for traumatic shock patients who arrive at major urban trauma centers has been reported to be 50%.

Another application area in which the measurement of oxygen saturation levels in tissue has proven useful is in fitness training. During training, such as for athletes using a bicycle ergometer where a cyclist is presented with increasing levels of work in stages, a point is reached in which the tissue oxygen saturation begins to drop below the established baseline. This is the breakpoint beyond which the muscle becomes increasingly hypoxic and transitions from aerobic metabolism to anaerobic metabolism. This is also the point at which serum lactate begins to rise above its established baseline and is known as the Lactate Breakpoint or Lactate Threshold. Studies show that endurance athletes achieve the highest performance when they do not exceed their Lactate Breakpoint during their weeks of training. Also, as athletes become more physically fit from training, this increase in fitness can be detected by means of an increase in their Lactate Breakpoint. Tissue oxygen saturation measurements can be used to determine the Lactate Breakpoint.

Existing ODIS systems are bulky an expensive, and therefore limited in utility for mobile applications such as fitness training and traumatic shock monitoring in settings including ambulance, helicopter, trauma center, Emergency Department (ED), or Intensive Care Units (ICU) of hospitals. In these settings, patients may be separated from physicians by considerable distances, and wireless transmission of oxygen saturation data allows early awareness of traumatic shock so that more timely decisions can be made regarding where the patient should be taken, and which medical staff members should be alerted to receive the patient. In both traumatic shock and fitness training applications, due to space limitations in mobile settings, the tissue oximeter must be as small as possible.

Therefore, what is needed is a miniaturized wireless tissue oximeter that is inexpensive to manufacture, and can be worn in mobile settings to wirelessly transmit immediate and continuous tissue oxygen saturation readings for both local and remote use.

### SUMMARY OF THE INVENTION

The present invention relates to a miniature wireless disposable optical tissue oximeter to monitor oxygen levels in tissue for use in shock trauma and exercise training applications. The oximeter measures local tissue oxygen saturation (SₜO₂) using near-infrared spectroscopy. The measurement is non-invasive, immediate and continuous.

In one embodiment the wireless disposable optical tissue oximeter consists of a wireless oximeter in a miniature form contained within an adhesive fixation unit and worn on the hand. The entire self-contained oximeter is very small in size, and therefore can be easily worn and used in ambulance, helicopter, trauma center, Emergency Department (ED), or Intensive Care Unit (ICU) of a hospital. The entire oximeter is disposable. The adhesive fixation unit is applied to the thenar eminence at the base of the thumb, and wrapped around to the back of the hand. The portion of the adhesive fixation unit that is located over the thenar eminence contains a sensor which in turn is connected to a programmable system on a chip. The system on a chip is powered by a battery, and communicates with a wireless transceiver and antenna unit.

In another embodiment, the adhesive fixation unit portion forms a disposable portion, and the remainder of the oximeter system forms a reusable portion for applications such as fitness training. A reusable sensor is removably attached the disposable adhesive fixation system, which has been adhesively applied over the muscle region to be trained, such as the calf or thigh. The reusable portion contains a sensor which in turn is connected by electrical cable to the remainder of the oximeter consisting of a programmable system on a chip which communicates with a wireless transceiver and antenna unit. The programmable system on a chip is powered by a battery. The reusable portion may be worn on the body by attachment means such as a belt, wrist band, leg band, or clothing clip. The programmable system on a chip calculates and transmits an easy to use consumer-friendly exercise index to allow athletes to adjust their exercise intensity level based on the present invention's non-invasive tissue oxygen saturation measurements rather than invasive blood lactate measurements. The exercise index value can be displayed numerically or in the form of an easy to understand red, yellow, and green light in which green indicates the intensity of exercise is in the aerobic range and exercise may continue, yellow indicates a transition from an aerobic to an anaerobic state and therefore exercise should be slowed, and red indicates the anaerobic range has been reached and that exercise should stop.

According to an aspect of the present invention, the sensor contains a first light source, a second light source, a photodetector, and a skin contact detector. Once skin contact is detected by means of the skin contact detector, the first light source emits light in the near infrared region, and the second light source emits light in the visible red region. The emitted light passes through a transparent layer of the adhesive fixation unit, and enters the underlying tissue, where a portion of the light is absorbed by tissue chromophores, including oxygenated hemoglobin and deoxygenated hemoglobin, and reflected back out of the tissue into the photodetector. The oxygen saturation of the tissue under the sensor is then calculated as the ratio of the measured concentration of the oxygenated hemoglobin divided by the total hemoglobin concentration, where the total hemoglobin concentration represents the sum of the measured oxygenated hemoglobin concentration and the measured deoxygenated hemoglobin concentration.

According to another aspect of the invention, the system on a chip monitors the skin contact sensor, and upon detection of skin contact automatically increases or decreases the intensity of the first and second light sources until the detector produces signals that are in the operating range. The first and second light sources are illuminated sequentially so that a corresponding detector measurement at each wavelength can be uniquely obtained. The system on a chip contains internal digital to analog converters that control the intensity of the first and second light sources, and also contains internal amplifiers and an analog to digital converter to obtain measurements from the photodetector. Furthermore the system on a chip contains a processor, read only memory, read-write memory, and a serial interface to communicate with the wireless transceiver. In addition, the system on a chip receives power from a miniature battery, and contains internal power conversion circuitry to provide supply voltages to the wireless transceiver.

According to yet another aspect of the invention, the oxygen saturation measurements are wirelessly transmitted to a remote display device, such as a smartphone running a smartphone software application which receives the measurements and displays them in numeric, graphical, and audible form. In addition, the smartphone software application may relay the data to the Internet for remote viewing on a web site or remote transfer to a hospital patient data system.

These and other advantages of the present invention will become apparent upon reading the following detailed descriptions and studying the various figures of the drawings.

### BRIEF DESCRIPTION OF THE DRAWINGS

The invention may best be understood by reference to the following description taken in conjunction with the accompanying drawings in which:

FIG. 1 is a block diagram representation of a wireless disposable shock trauma monitoring device in accordance with an embodiment of the present invention.

FIG. 2A is a diagrammatic top view representation of a wireless disposable shock trauma monitoring device in accordance with an embodiment of the present invention.

FIG. 2B is a diagrammatic side view representation of a wireless disposable shock trauma monitoring device in accordance with an embodiment of the present invention.

FIG. 3 is a perspective view of a wireless disposable shock trauma monitoring device placed on a hand.

FIG. 4 is a perspective view of a wireless disposable shock trauma monitoring device placed on the calf of a leg.

FIG. 5 is a process flow diagram in accordance with an embodiment of the present invention.

FIG. 6 is a time-course plot of tissue oxygen saturation in accordance with an embodiment of the present invention.

FIG. 7 is a plot of blood lactate as a function of running speed before and after physical training in accordance with an embodiment of the present invention.

FIG. 8 is a plot of breakpoint workload derived from tissue oxygen saturation versus breakpoint workload derived from lactate in accordance with an embodiment of the present invention.

FIG. 9A is a plot of a first exercise index versus exercise stage in accordance with an embodiment of the present invention.

FIG. 9B is a plot of a second exercise index versus exercise stage in accordance with an embodiment of the present invention.

FIG. 9C is a plot of a third exercise index versus exercise stage in accordance with an embodiment of the present invention.

FIG. 9D is a plot of a fourth exercise index versus exercise stage in accordance with an embodiment of the present invention.

FIG. 9E is a plot of a fifth exercise index versus exercise stage in accordance with an embodiment of the present invention.

FIG. 10A is a plot of a first exercise index versus lactate in accordance with an embodiment of the present invention.

FIG. 10B is a plot of a second exercise index versus lactate in accordance with an embodiment of the present invention.

FIG. 10C is a plot of a third exercise index versus lactate in accordance with an embodiment of the present invention.

FIG. 10D is a plot of a fourth exercise index versus lactate in accordance with an embodiment of the present invention.

FIG. 10E is a plot of a fifth exercise index versus lactate in accordance with an embodiment of the present invention.

### DETAILED DESCRIPTION OF THE EMBODIMENTS

The present invention relates to a miniature wireless disposable optical tissue oximeter to monitor oxygen levels in tissue for use in shock trauma and exercise training applications. The oximeter measures local tissue oxygen saturation (SₜO₂) using near-infrared spectroscopy. The measurement is non-invasive, immediate and continuous.

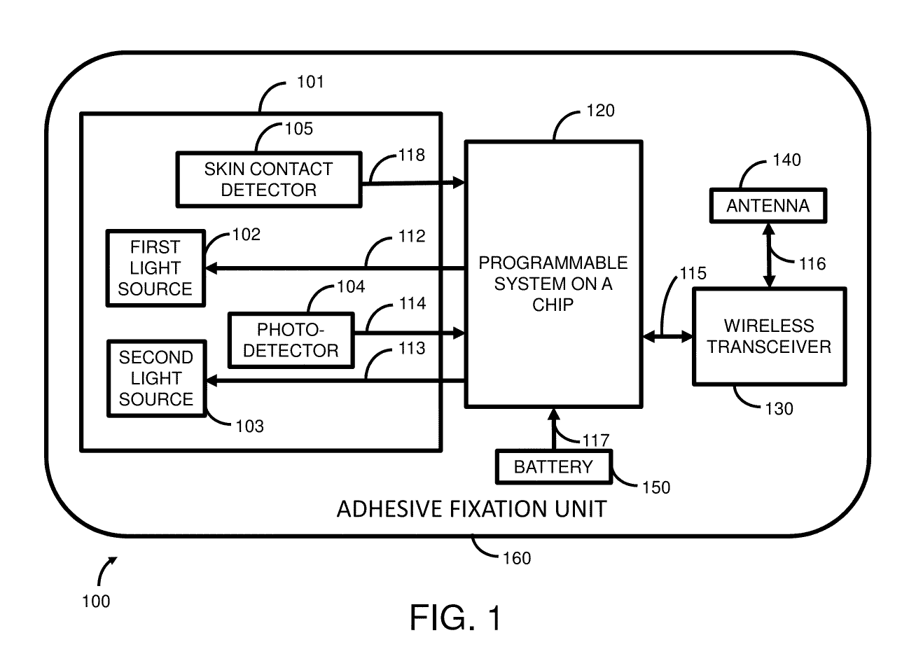

FIG. 1 is a block diagram representation of a wireless disposable shock trauma monitoring system 100 in accordance with an embodiment of the present invention consisting of sensor 101 which contains a first light source 102, a second light source 103, and photodetector 104. The first light source 102 emits a first beam of light in the near infrared region into the tissue, and the second light source 103 emits a second beam of light in the visible red region into the tissue. By way of example, the first light source may emit at a wavelength of 905 nm and the second light source may emit at a wavelength of 660 nm. It should be appreciated, however, that the wavelengths of light produced by light emitting diodes associated with first light source 102 and second light source 103 may vary widely. The first beam of light and the second beam of light enter the tissue, and a portion of each beam is reflected by the tissue and received by photodetector 104. In addition, sensor 101 contains skin contact detector 105. By way of example, skin contact detector 105 may consist of a planar conductive element forming a first plate of a capacitor, adjacent to one or more conductive elements forming a second plate of a capacitor. Skin contact detector 105 is electrically insulated from the skin by means of adhesive fixation unit 160. The total capacitance value between the first plate and the second plate is increased by contact with human skin which serves as an electrical dielectric, and therefore measurement of the capacitive value allows for the detection of skin contact. The skin contact detector is located near first light source 102, second light source 103, and photodetector 104 to detect contact with the skin.

Sensor 101 interconnects with a programmable system on a chip (PSOC) 120. These connections consist of connection 112 joining the first light source 102 to PSOC 120 through which the PSOC can control the intensity of the first light source, connection 113 joining the second light source 103 to PSOC 120 through which the PSOC can control the intensity of the second light source, connection 114 joining the photodetector 104 to PSOC 120 through which the PSOC can measure the electrical signal from photodetector 104, and connection 118 joining skin contact detector 105 to PSOC 120 through which the PSOC can detect whether or not the sensor 101 is in contact with skin.

The programmable system on a chip 120 receives power from battery 150 by means of connection 117. The programmable system on a chip 120 transmits data including tissue oxygen saturation measurements to wireless transceiver 130, which in turn transmits and receives information from antenna 140 by means of connection 116. The system on a chip 120 contains internal digital to analog converters that control the intensity of the first and second light sources, and also contains internal amplifiers and an analog to digital converter to obtain measurements from the photodetector. Furthermore the system on a chip 120 contains a processor, read only memory, read-write memory, and a serial interface to communicate with the wireless transceiver. In addition, the system on a chip 120 receives power from a miniature battery, and contains internal power conversion circuitry to provide supply voltages to the wireless transceiver 130. An example of such a programmable system on a chip is the Cypress Semiconductor PSoC® 5 CY8C55. An example of such a wireless transceiver with connected antenna is the Roving Networks RN42 Bluetooth Transceiver module.

Adhesive fixation unit 160 contains all of the system components including sensor 101, programmable system on a chip 120, battery 150, wireless transceiver 130, antenna 140 and their associated interconnections, thereby forming a fully self-contained disposable miniature oximeter system.

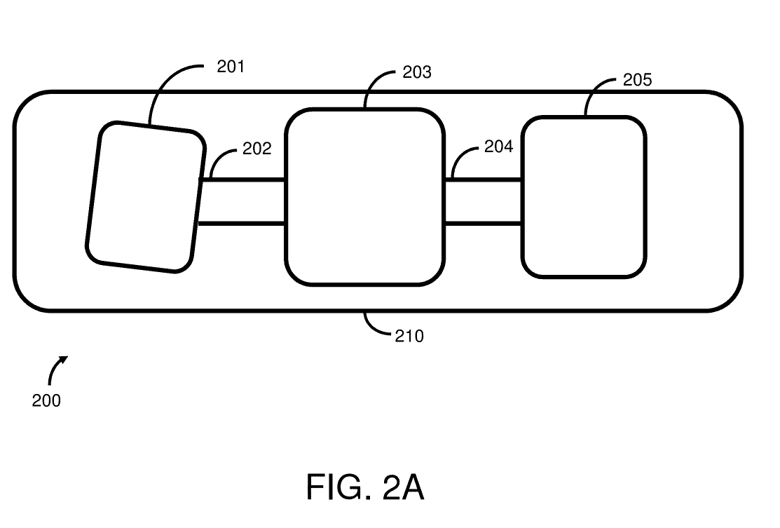

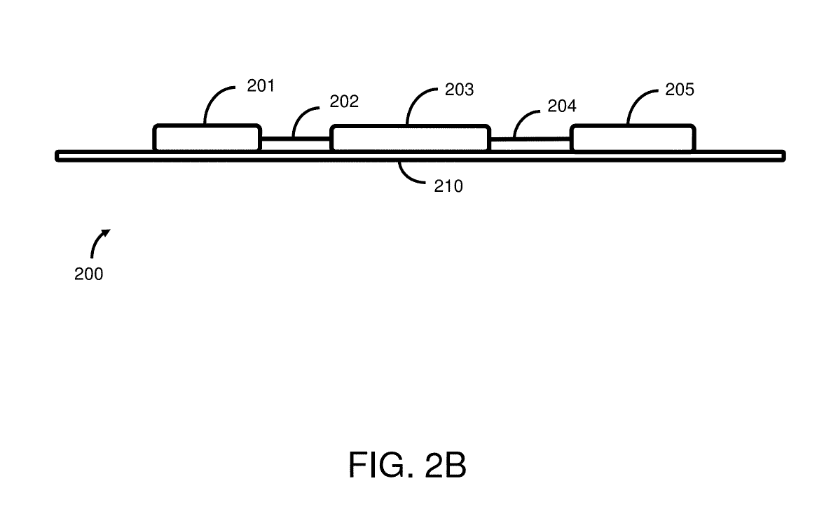

FIG. 2A is a diagrammatic top view representation of a wireless disposable shock trauma monitoring device 200 in accordance with an embodiment of the present invention. Opaque compartment 201 houses sensor 101 and secures it to biocompatible transparent pressure sensitive adhesive film 210. Compartment 203 houses the programmable system on a chip 120 and battery 150 and secures it to the transparent pressure sensitive adhesive film 210. Compartment 205 houses the wireless transceiver 130 and antenna 140 and secures it to the transparent pressure sensitive adhesive film 210. Compartment 201 is electrically connected to compartment 203 by means of flexible connection 202. Compartment 203 is electrically connected to compartment 205 by means of flexible connection 204. An example of such transparent pressure sensitive adhesive film is Scapa RX1402P single coated pressure sensitive adhesive biocompatible 0.003 inch thick polyethylene film. An example of material for compartment 201, compartment 203, and compartment 205 is Scapa 0399003 single coated pressure sensitive adhesive biocompatible ⅛ inch thick polyethylene foam, covered by an outer opaque layer of Scapa RX848P biocompatible metallized polypropylene film.

FIG. 2B is a diagrammatic side view representation of a wireless disposable shock trauma monitoring device 200 in accordance with an embodiment of the present invention. The emitted light from first light source 102 and second light source 103 within sensor compartment 201 passes through a transparent layer 210 of the adhesive fixation unit, and enters the tissue upon which adhesive fixation layer 210 has been applied, where a portion of the light is absorbed by tissue chromophores, including oxygenated hemoglobin and deoxygenated hemoglobin, and reflected back out of the tissue into photodetector 104 contained within compartment 201. The oxygen saturation of the tissue under the sensor is then calculated as the ratio of the measured concentration of the oxygenated hemoglobin divided by the total hemoglobin concentration, where the total hemoglobin concentration represents the sum of the measured oxygenated hemoglobin concentration and the measured deoxygenated hemoglobin concentration.

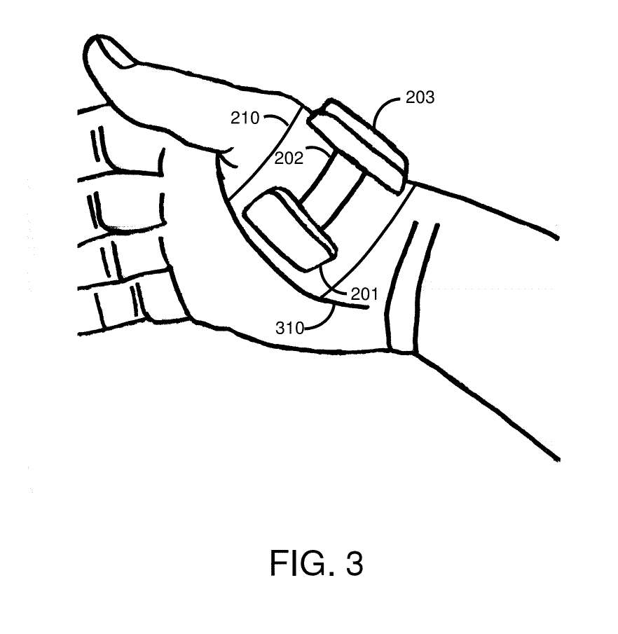

FIG. 3 is a perspective view of a wireless disposable shock trauma monitoring device placed on and secured to a hand. Transparent pressure sensitive adhesive film 210 is positioned and adhesively secured to the hand such that the long axis of sensor compartment 201 is aligned with the long axis of the thenar eminence 310 of the hand and compartment 203 containing the programmable system on a chip and battery is positioned over the dorsal aspect of the back of the hand. Compartment 201 is electrically connected to compartment 203 by means of flexible connection 202.

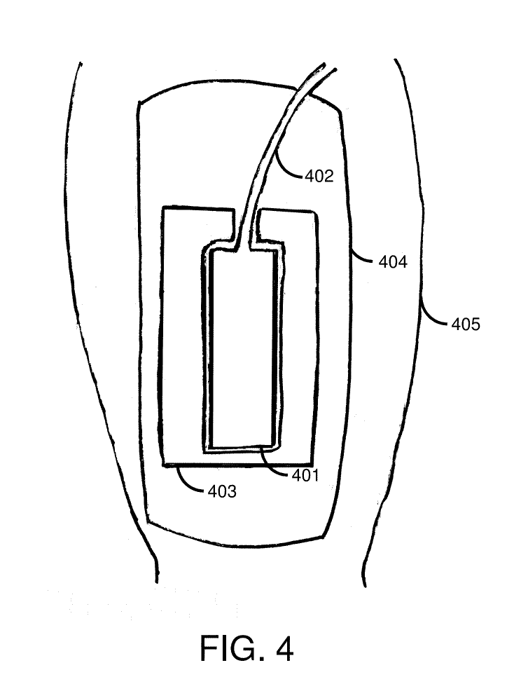

FIG. 4 is a perspective view of another embodiment of the present invention, in which reusable sensor element 401 containing sensor 101 is removably affixed to compartment 403. Compartment 403 is permanently affixed to transparent pressure sensitive adhesive film 404 which is adhesively applied the calf of a leg. Reusable sensor element 401 is electrically connected to the programmable system on a chip 120 by means of electrical cable 402. Compartment 403 and transparent pressure sensitive adhesive film 404 form a disposable adhesive fixation unit.

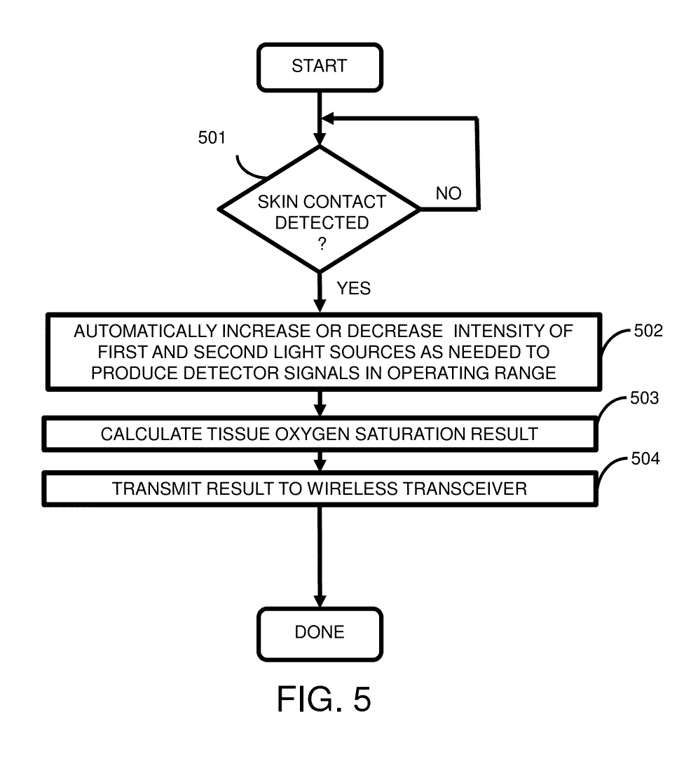

FIG. 5 is a process flow diagram in accordance with an embodiment of the present invention, illustrating one method by which the programmable system on a chip 120 can obtain and wirelessly transmit oxygen saturation values. The process begins at step 501 in which the programmable system on a chip 120 determines whether or not contact with the skin has been detected by means of measurements obtained from skin contact detector 105. If skin contact has not been detected, step 501 is returned to until skin contact is detected. When skin contact has been detected, the process proceeds to step 502 in which the intensity of first light source 102 and second light source 103 are automatically increased or decreased as needed to produce detector signals that are within the operating range of photodetector 104. The process then proceeds to step 503 in which the tissue oxygen saturation value is calculated based on readings obtained from photodetector 102. The process finally proceeds to step 504 in which the tissue oxygen saturation results are transmitted to the wireless transceiver.

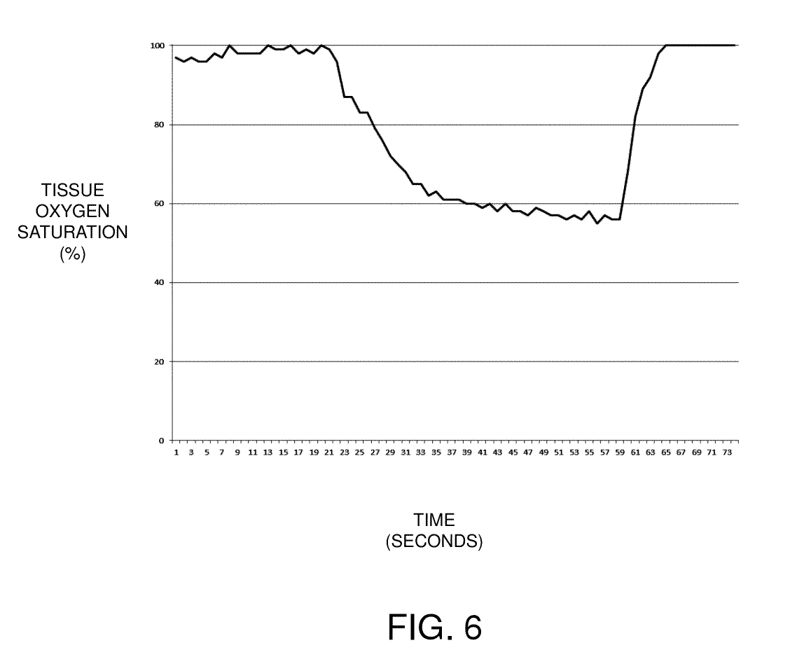

FIG. 6 is a time-course plot of tissue oxygen saturation obtained from a prototype of one embodiment of the present invention that has been reduced to practice. Sensor 101 was placed on the thenar eminence of a human hand. The vertical axis represents calculated tissue oxygen saturation in percent units, and the horizontal axis represents elapsed time in seconds. Baseline measurements obtained during the first 20 seconds demonstrate initial tissue oxygen saturation readings between 95% to 100%. Pressure was then applied to the tissue of the hand, thereby reducing blood perfusion and was maintained for 40 seconds. During this period of applied pressure, the measured tissue oxygen saturation values steadily declined to under 60%. When the applied pressure was removed, circulation in the tissue under the sensor was therefore restored and a corresponding rise in tissue oxygen saturation was measured reaching 100%. FIG. 5 therefore demonstrates that the present invention is sensitive to changes in tissue perfusion.

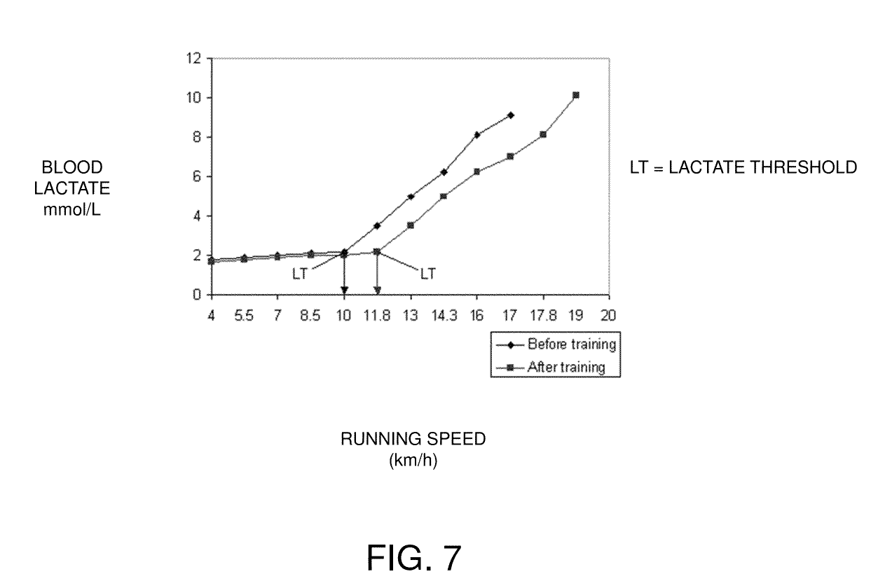

FIG. 7 is a plot of blood lactate as a function of running speed before and after physical training in accordance with an embodiment of the present invention. In fitness training, runners may be placed on a bicycle ergometer where they are presented with increasing levels of work in stages. As the level of work increases, a point is reached in which the tissue oxygen saturation begins to drop below an established baseline. This point represents the “breakpoint” beyond which the muscle becomes increasingly hypoxic and transitions from aerobic metabolism to anaerobic metabolism. This is also the point at which the lactate begins to rise above its established baseline and is known by those skilled in the art as the “Lactate Breakpoint” or “Lactate Threshold (LT)”.

Studies show that endurance athletes achieve the highest performance when they do not exceed their “Lactate Threshold” during their many weeks of training. For an ordinary person interested in fitness, it would therefore be useful to be alerted to when their muscles are becoming hypoxic during exercise so that they can adjust their level of exertion to match their own threshold. Also, as athletes become more physically fit from training, this increase in fitness can be detected by means of an increase in their LT.

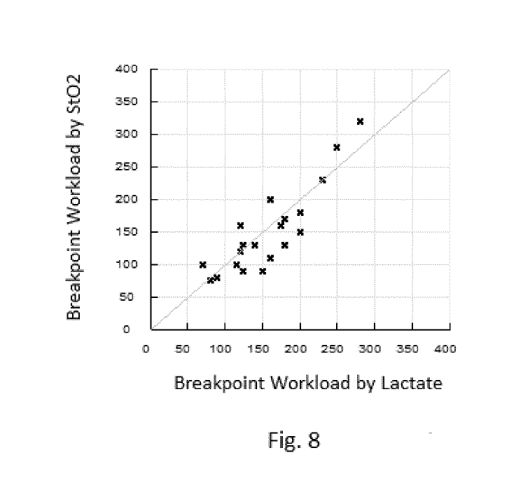

FIG. 8 is a plot of breakpoint workload derived from tissue oxygen saturation versus breakpoint workload derived from lactate in accordance with an embodiment of the present invention. This plot was obtained from the literature, and demonstrates that the breakpoint workload as measured by blood lactate measurements correlates with the breakpoint workload as obtained by tissue oxygen saturation measurements.

An embodiment of the present invention provides an easy to use consumer-friendly index for exercise intensity level based on non-invasive tissue oxygen saturation measurements rather than invasive blood lactate measurements. Let StO₂ be the current value of tissue oxygen saturation provided by a tissue oximeter in the unit of percentage (%) and StO₂|_{At rest} be the StO₂ reading at rest before exercise, also called baseline for StO₂. Then we define the following five parameters as candidates for exercise indices based on StO₂. All these exercise indices are unitless and range from 0 to 100, as follows:

### Exercise Index Ox-1 (or “Index for Muscle Oxygen Level”)

Ox-1=100−StO₂. (1)

### Exercise Index Ox-2 (or “Index for Muscle Oxygen Drop Rate”)

Ox-2=[Drop Rate of StO₂]×5. (2)

Here the unit of StO₂ drop rate is percentage per hour. We apply the multiplying factor 5 because StO₂ drop rates ≧20%/hour are observed when blood supply to a tissue flap is blocked.

### Exercise Index Ox-3 (or “Index for Oxygen Ratio”, or “Anaerobic Index 1”)

Ox-3=(StO₂|_{At rest}/StO₂−1)×100. (3)

### Exercise Index Ox-4 (or “Index for Oxygen Difference”, or “Anaerobic Index 2”)

Ox-4=StO₂|_{At rest}−StO₂. (4)

### Exercise Index Ox-5 (or “Combined Exercise Index”)

Ox-5=Maximum of Indices Ox-1,Ox-2,Ox-3 and Ox-4. (5)

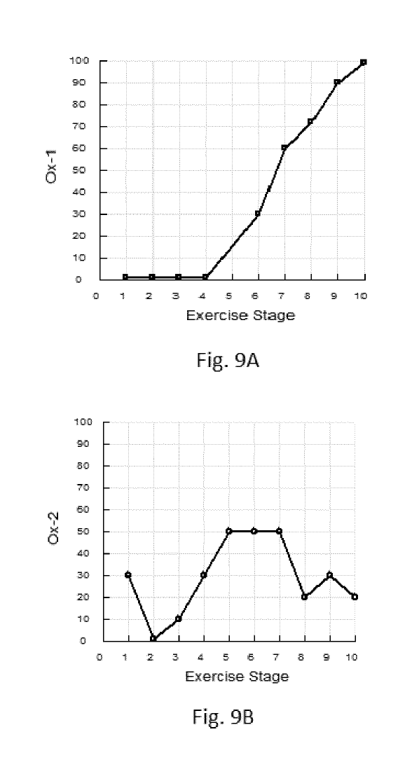

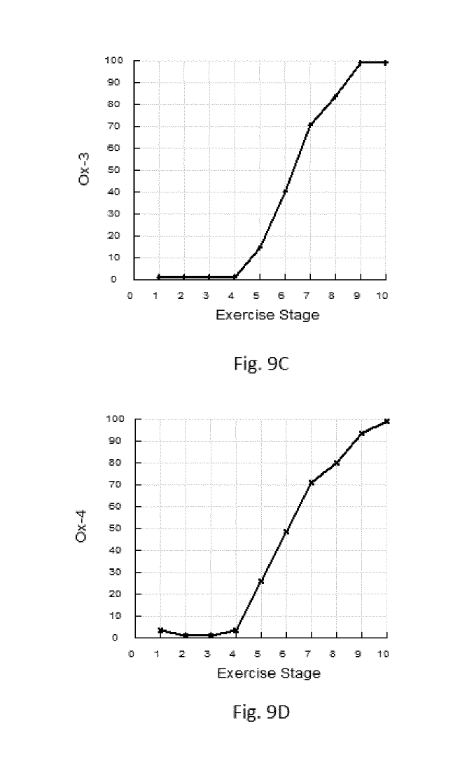

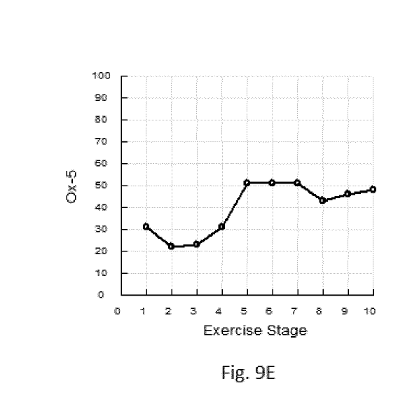

FIGS. 9A, 9B, 9C, 9D, and 9E are plots of exercise index Ox-1, Ox-2, Ox-3, Ox-4, and Ox-5 respectively versus exercise stage in accordance with an embodiment of the present invention. Values of these five exercise indices are graphically shown at different stages of exercise. In the calculation of StO₂ drop rate the time duration employed for each stage is 30 minutes. Statistical parameters for time series of the indices are listed in Table 1. All exercise indices at rest before exercise are of a small value close to zero. As the oximeter user exercises, the StO₂ of the muscle decreases, and all the exercise indices, as well as the lactate value, rise as a trend. Therefore the exercise indices are correlated to the Lactate value, and both remain near their own baselines until the muscle becomes hypoxic, transitioning from aerobic metabolism to anaerobic metabolism. In addition, both parameters rise as a trend when exercise intensity increases.

TABLE 1
Comparison between the five exercise indices.

| Exercise Index | Baseline required | R² | p-value |
|---|---|---|---|
| Ox-1 | No | 0.92 | 0.22 |
| Ox-2 | No | 0.61 | 0.21 |
| Ox-3 | Yes | 0.92 | 0.20 |
| Ox-4 | Yes | 0.92 | 0.13 |
| Ox-5 | Yes | 0.98 | 0.22 |

Consider the following linear calibration for the five exercise indices”

Ox-i=Ox-i×k+b, i=1,2,3,4,5. (6)

Here k and b are linear calibration factors. When the calibration factors are as listed in Table 2 below, the calibrated exercise indices are graphically shown in FIG. 9.

TABLE 2
Factors for linear calibration.

| Exercise Index | k | b |
|---|---|---|
| Ox-1 | 6 | −180 |
| Ox-2 | 1 | 0 |
| Ox-3 | 3 | −20 |
| Ox-4 | 4.5 | −10 |
| Ox-5 | 1 | 0 |

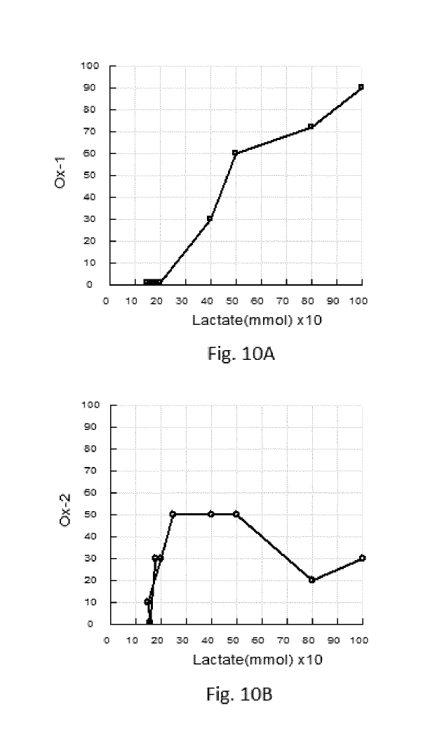

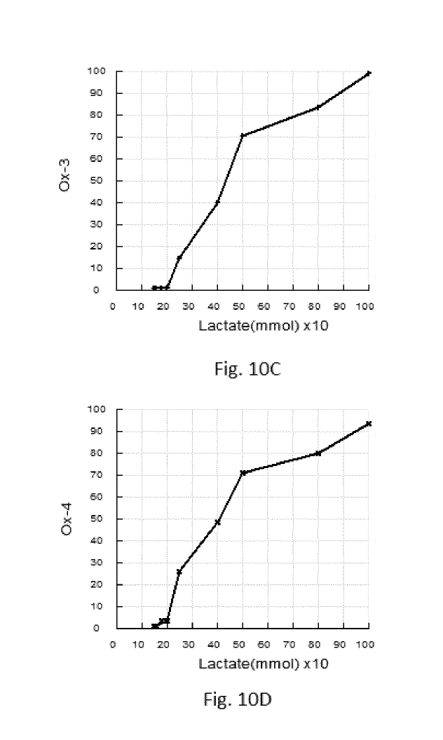

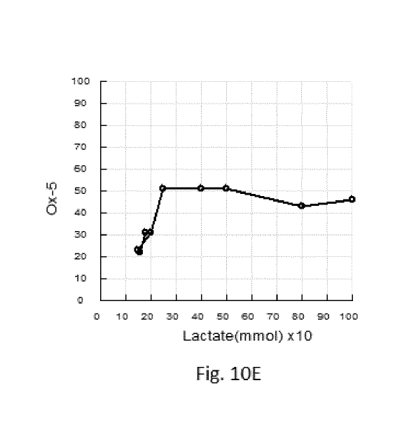

FIGS. 10A, 10B, 10C, 10D, and 10E are plots of exercise index Ox-1, Ox-2, Ox-3, Ox-4, and Ox-5 respectively versus lactate in accordance with an embodiment of the present invention.

In an embodiment of the present invention, an exercise index value can be displayed numerically or in the form of an easy to understand red, yellow, and green light in which green indicates the intensity of exercise is in the aerobic range and exercise may continue, yellow indicates a transition from an aerobic to an anaerobic state and therefore exercise should be slowed, and red indicates the anaerobic range has been reached and that exercise should stop.

The present examples are to be considered as illustrative and not restrictive, and the invention is not to be limited to the details given herein, but may be modified within the scope of the appended claims.

What is claimed is:

1. A tissue oximeter comprising:
a first light source, the first light source being arranged to provide a first beam of light;
a second light source, the second light source being arranged to provide a second beam of light;
a photodetector, the photodetector being arranged to receive reflected light from the first light source and the second light source;
a skin contact detector, the skin contact detector being arranged to detect when the first light source, the second light source, and the photodetector are in contact with the skin;
a programmable system on a chip, the programmable system on a chip configured to control the intensity of the first light source and the second light source, to measure the output of the photodetector, to measure the output of the skin contact sensor, and to compute oxygen saturation values based on photodetector measurements;
a battery, the battery being arranged to provide power to the programmable system on a chip;
a wireless transceiver, the wireless transceiver being arranged to transmit the oxygen saturation values from the system on a chip; and
a disposable adhesive fixation system, the adhesive fixation system arranged to fully contain the tissue oximeter and adhere it to the skin.

2. The tissue oximeter of claim 1 wherein the first light source emits near infrared light at a wavelength of 905 nm.

3. The tissue oximeter of claim 1 wherein the second light source emits visible red light at a wavelength of 660 nm.

4. The tissue oximeter of claim 1 wherein the skin contact detector measures the capacitance of a region under the detector.

5. The tissue oximeter of claim 1 wherein the adhesive fixation system includes a biocompatible transparent film to separate the first light source, second light source, photodetector, and skin contact detector from the skin.

6. The tissue oximeter of claim 1 wherein the adhesive fixation system includes first compartment to house the first light source, second light source, photodetector, and skin contact sensor, the first compartment being oriented to align with the thenar eminence of the hand, a second compartment to house a programmable system on a chip and battery, and a third compartment to house a wireless transceiver.

7. The tissue oximeter of claim 1 wherein the adhesive fixation system includes first compartment to house the first light source, second light source, photodetector, and skin contact sensor, the first compartment being oriented to align with the thenar eminence of the hand, and a second compartment to house a programmable system on a chip, a battery, and a wireless transceiver.

8. The tissue oximeter of claim 1 wherein the adhesive fixation system includes a single compartment to house the first light source, second light source, photodetector, skin contact sensor, system on a chip, a battery, and a wireless transceiver, the single compartment being oriented to align with the thenar eminence of the hand

9. A tissue oximeter comprising:
a first light source, the first light source being arranged to provide a first beam of light;
a second light source, the second light source being arranged to provide a second beam of light;
a photodetector, the photodetector being arranged to receive reflected light from the first light source and the second light source;
a skin contact detector, the skin contact detector being arranged to detect when the first light source, the second light source, and the photodetector are in contact with the skin;
a programmable system on a chip, the programmable system on a chip configured to control the intensity of the first light source and the second light source, to measure the output of the photodetector, to measure the output of the skin contact sensor, and to compute oxygen saturation values based on photodetector measurements;
a battery, the battery being arranged to provide power to the programmable system on a chip;
a wireless transceiver, the wireless transceiver being arranged to transmit the oxygen saturation values from the system on a chip; and
a disposable adhesive fixation system, the adhesive fixation system arranged to be removably attachable to a portion of the tissue oximeter and adhere it to the skin of the lower or upper extremeties.

10. The tissue oximeter of claim 9 wherein the first light source emits near infrared light at a wavelength of 905 nm.

11. The tissue oximeter of claim 9 wherein the second light source emits visible red light at a wavelength of 660 nm.

12. The tissue oximeter of claim 9 wherein the skin contact detector measures the capacitance of a region under the detector.

13. The tissue oximeter of claim 9 wherein the adhesive fixation system includes a biocompatible transparent film to separate the first light source, second light source, photodetector, and skin contact detector from the skin.

14. The tissue oximeter of claim 9 wherein the disposable adhesive fixation system includes a compartment in which the first light source, second light source, photodetector, and skin contact sensor can be removably detached from the adhesive fixation system for reuse.

15. The tissue oximeter of claim 9 wherein the programmable system on a chip computes an exercise index as a function of the computed oxygen saturation values such that the exercise index provides a surrogate for serum lactate concentration.
